(19) United States
(12) Patent Application Publication
(10) Pub. No.: US 2007/0282197 A1
(43) Pub. Date: Dec. 6, 2007

(54) Instrument, imaging position fixing system and position fixing method

(75) Inventors: Ulrich Bill, Effeltrich (DE); Martin Hoheisel, Erlangen (DE)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Aktiengesellschaft

(21) Appl. No.: 11/803,376

(22) Filed: May 14, 2007

(30) Foreign Application Priority Data
May 19, 2006 (DE) 10 2006 023 733.1

(51) Int. Cl. A61B 5/05; A61B 8/14

(52) U.S. Cl. 600/424; 600/467; 128/899; 600/407

(57) ABSTRACT

The invention relates to an instrument able to be introduced into a body as well as an imaging position fixing system and position fixing method for the instrument. A magnet able to be rotated with a rotation drive is provided in an end section of the instrument. The position of the magnet in the body can be determined on the basis of the strength of a magnetic alternating field created by a rotation of the magnet.

### CROSS REFERENCE TO RELATED APPLICATIONS

This application claims priority of German application No. 10 2006 0223 filed May 19, 2006, which is incorporated by reference herein in its entirety.

### FIELD OF THE INVENTION

The invention relates to an instrument, an imaging position fixing system and an imaging position fixing method for the instrument.

### BACKGROUND OF THE INVENTION

A marker which can be moved within a hollow cavity of a body of a living being is known from DE 195 32 676 C1. The marker is made from a magnetic material. To determine the position of the marker a pulsed external magnetic field is applied to it from a predetermined direction. To create the magnetic field a coil system is provided. A magnetic remanence field is created by the magnetic field in the marker in the predetermined direction. The strength of the magnetic remanence field is measured in the absence of the external magnetic field by means of magnetic coils. The position of the marker in the body is determined on the basis of the strength of the remanence field. A disadvantage of the known method for determining the position is that to the coil system provided for magnetizing the marker is complex to construct. It is further necessary to magnetize the marker periodically. This necessitates a complex negative phase triggering of the coil system and the measurement coils. Furthermore the accuracy of the position determination is adversely affected by the decaying of the remanence field.

### SUMMARY OF THE INVENTION

The object of the invention is to overcome the disadvantages of the prior art. In particular an instrument is to be provided of which the position in a body can be determined with particular accuracy, with high sensitivity and in a simple manner. A further object of the present invention is to provide an imaging position fixing system and an imaging position fixing method which allows an especially accurate, highly sensitive and simple fixing of the position of the instrument in the body.

This object is achieved by the features of the claims.

According to the invention an instrument is provided with a free first end for introduction into a body, in which case, for determination of a position of the instrument, a magnet is provided in an end section containing the free end, said magnet being connected to a rotation drive to allow it to rotate. The rotation drive is provided to rotate the magnet and is connected to the latter. As a result of the rotation the magnet generates an alternating magnetic field. The higher the frequency of the rotation of the magnet is the greater is the strength of the alternating field created. The term “strength of the alternating field” can be understood to mean a speed of change of a magnetic field, a slew rate or a similar variable. By selecting a suitably high rotational frequency of the rotation drive and thereby of the magnet, the strength of the alternating field can be set such that it is possible to position the magnet on the basis of the strength of the alternating field detected outside the body. The alternating field can be created with a strength which enables direction and position changes of the end section to be determined with high accuracy. This enables an especially precise and highly-sensitive position fixing and determination of the position of the magnet in the body. Furthermore the inventive principle of the rotatable magnet accommodated in the end section can be implemented for a plurality of known instruments in an especially simple manner. In addition, if the geometry of the instrument, e.g. its length and/or thickness, and a series of positions of the magnet in the body are used, the instrument and its position in the body can also be shown.

The body involved can be any given body. It can especially be the body of a living being, especially of a human being, or of a living being of which the body is comparable in size with that of mammals.

The instrument can for example be used in inanimate bodies for non-destructive material testing. With living beings the instrument can be used for non-invasive or minimally-invasive investigations of the section of the inside of the body.

The term rotation drive is especially understood as a drive for creating and/or transmitting a rotational movement.

The magnet can be a permanent magnet. It is also possible for the magnet to be an electromagnet. To supply the electromagnet with electrical energy lines can be provided routed within the instrument. The energy can be transmitted from the lines to the electromagnet by wiper contacts for example.

According to an embodiment of the instrument there is provision for the rotation drive to include a shaft, preferably a flexible shaft, brought out at the second end of the instrument, for transmitting to the magnet a rotational movement of a drive unit provided outside the instrument. The magnet can be attached directly to the shaft so that can rotate around its axis. The magnet can also be accommodated on an attachment arm fixed to the shaft to allow rotation. A bearing can also be provided. This allows an especially precise rotation of the magnet around the support axis to be achieved. According to an especially advantageous embodiment, a shaft, which is typically provided in any event in a medical instrument to transmit a rotational movement, such as an OCT or IVUS catheter for example, can be used as the shaft to accommodate the magnet.

The shaft can be guided in the instrument in a tube, preferably a flexible tube. The tube makes is possible to guide the shaft especially precisely in the instrument. Friction losses during rotation can be significantly reduced by a suitable surface property of the inner surface of the tube and/or the outer surface of the shaft. A disproportionate torsional stress on the shaft and wear on the tube can also be greatly reduced. The magnet can by rotated at an especially constant frequency at a predetermined drive torque. In this way the accuracy of determining the position of the magnet is further improved.

Preferably the shaft and/or the tube is/are made of a plastic material. The shaft and/or the tube can however also be made of any given flexible material which has a suitable tearing strength, bending strength and torsional strength in order to rotate the magnet at a constant frequency. With flexibly embodied instruments, such as catheters for example, the material is preferably flexible and has sufficient bending strength to enable the instrument to be bent without damage to the shaft and/or the tube in accordance with the relevant requirements. This provides a guarantee of a constant and precise rotation of the magnet without the adverse effects of a bending of the instrument and enables its position to be determined exactly.

In accordance with an embodiment of the invention the rotation drive includes the drive unit. To establish a positive connection with the shaft the drive unit can feature a clutch. The provision of the clutch makes it simple to connect and disconnect the shaft from the drive unit. The clutch can have cylinders or rollers between which the shaft can be accommodated with a friction fit. By turning the cylinders or rollers a rotation force acting tangentially to a longitudinal direction of the shaft can be transmitted. The clutch concerned can also be a flange clutch, floating clutch, claw clutch, magnetic clutch or another similar clutch or clutch operating in the same manner.

According to an advantageous embodiment of the invention the rotation drive includes a drive unit provided in the end section. In this case the instrument can be used independently of an external drive unit. To supply the drive unit with energy lines routed in the instrument can be provided, which are brought out of the instrument at the second end and can be connected to an external energy supply. With a drive unit operated by means of electrical energy an accumulator can also be provided within or outside the instrument for energy supply.

The drive unit provided outside the instrument or in its end section can be an electric motor, a stepping motor, a piezoelectric motor, a turbine motor, a hydraulic motor or a pneumatic motor. The different motor types can be selected to meet particular requirements, such as torque, rotation frequency, size, compatibility with medical devices and materials etc. . . . For example especially small sizes can be achieved with piezoelectric motors. Piezoelectric motors and turbine motors can for example be made exclusively from ceramics and plastics. As a result of the plurality of possible motor types the magnet can be accommodated in the end section of a plurality of instruments of different shapes and sizes and can be provided with a drive unit.

In accordance with an embodiment of the invention there is provision for the rotation drive to include a gear. The gear involved can be a reduction gear or step-up gear. The gear can be provided outside the instrument or also in the end section between of the drive unit and the shaft or between the shaft and the magnet. The gear makes it possible in a simple manner to convert the rotary frequency of the drive unit or shaft into a rotation frequency of the magnet suitable for creating an alternating field of the magnet with sufficient strength.

An embodiment of the invention provides for an ultrasound converter for performing intravascular ultrasound investigations able to be rotated by means of the rotation drive to be provided in the area of the end section. A mirror for performing optical coherence tomography examinations, able to be rotated by means of the rotation drive, can also be provided in the area of the end section. No separate drives are required for the ultrasound converter or mirror. They can be connected to the rotation drive just like the magnet. This allows an especially simple construction and a small size to be achieved.

The instrument can be an instrument selected from the following group of medical instruments: Catheters, especially IVUS or OCT catheters, needles, especially puncturing needles or biopsy needles, probes, especially stomach or bowel probes. In addition to the rotation of the magnet the rotation drive can fulfill further functions with the above-mentioned instruments such as for example a rotation of the ultrasound converter or mirror in the IVUS or OCT catheter.

In accordance with a further aspect of the invention an imaging position fixing system for determining the position of the instrument introduced into a body in accordance with the invention can be provided, including:
An image recording device for recording image data for generation of a first image of a section of the inside of the body, where the image data is correlated with first coordinates of a first coordinate system defined by the image recording device,
A magnetic field detection device arranged outside the body with at least one sensor for detecting a strength of an alternating field created by the rotation of the magnet in the body,
A position determination device for determining a position of the magnet based on the strength of the detected alternating field, with the position of the magnet being correlated with second coordinates of a second coordinate system defined by the magnetic field device,

A correlation device for correlation of the first coordinate system with the second coordinate system and
An image generation device for creating the first image reproducing the section of the body and a second image overlaid on it reproducing the position of the magnet.

The proposed position fixing system is suitable for determining the position of a magnet rotating in an instrument by means of the rotation drive. The strength of the alternating field created by the rotation of the magnet can be detected with the at least one sensor of the magnetic field detection unit. To allow an especially precise position fixing of the magnet in the body it is necessary for the alternating field to also exceed a minimum field strength able to be detected by the sensor outside the body. To achieve this a suitable high value for the rotation frequency of the magnet can be selected by for example using an electric motor with a correspondingly high speed and/or a step-up gear. The strength of the alternating field detected with the sensor is, regardless of the rotation frequency, dependent on the distance of sensor from the magnet and the direction of the axis of rotation of the magnet. A directional dependency of the alternating field can be taken into account with anisotropic sensors. Using the distance dependency, conclusions can be drawn about the position of the magnet in the body based on the detected strength of the alternating field. Because the strength of the alternating field depends on the rotation frequency it is necessary, to ensure an especially precise determination of the position of the magnet, for the magnet to be rotated at an essentially constant rotation frequency. This can be achieved in an advantageous manner with the inventive instrument which is expediently a component of the position fixing system.

To further increase the accuracy of determining the position of the magnet the magnetic field detection device can include a number of sensors arranged separated spatially from one another. With a number of sensors a plurality of non-redundant information about the distance of the sensors from the magnet and the position of the magnet can be determined. This enables the position of the magnet to be determined especially quickly, accurately and uniquely.

With a number of sensors the position of the magnet can be determined using a similar method to that known from DE 195 32 676 C1. In this case the magnetic field detection unit can feature at least one pair of anisotropic sensors which are able to be positioned on opposite sides of the body. The sensors feature sensor surfaces which make it possible to detect the strength of components of the alternating field in parallel and/or at right angles to the axis joining the pair of sensors. The magnetic field detection device can include one or more controllers and/or a computer. As many sensors can be provided as are necessary for especially precise determination of the position of the magnet. For example 1 to 3, 3 to 10, 10 to 30, 30 to 60, 60 to 100 or more sensors can be provided.

The position determination unit is provided for determining the position of the magnet. Expediently the position determination unit includes a computer with which the position of the magnet can be determined on the basis of the strength of the alternating field detected by the sensors. To determine the position the alternating field can be measured by means of the sensors. On the basis of the signals created by the sensors, using predetermined algorithms, with which for example disturbances also caused by electrical conductors, ferromagnetic objects and such like can be taken into account, the position of the magnet can be determined. The position of the magnet determined by the position determination unit can be described in a simple manner by second coordinates in a second coordinate system defined by the magnetic field detection unit.

The correlation device is provided for correlation of the first coordinate system with the second coordinate system. The image data, e.g. the individual pixels of an x-ray image, can be described by first coordinates in the first coordinate system defined by the recording device. The first and second coordinate system are preferably three-dimensional coordinate systems. Such systems can be Cartesian, cylinder or sphere coordinate systems.

As a result of the correlation it is possible in a simple manner to convert into first coordinates of the first coordinate system coordinate points described by second coordinates in the second coordinate system, such as the position of the magnet, for example. After a correlation of the first and second coordinate system an overlay image can be created by the image generation device, which contains the first image reproducing the body section and the second image reproducing the position of the magnet. The second image can be a simple graphical presentation, such as a cross, a point, an arrow or suchlike. It is also possible for the second image to include a presentation of at least the end section of the instrument. In the case of an introduction of the instrument into the body, a track reflecting the path of the instrument in the body can be shown.

With a change in the position of the magnet, e.g. as a result of a displacement of the instrument relative to the body, the position of the magnet can be determined once again. Subsequently the overlay image can be updated. In this case the image data already present or the first image present can be used. It is not necessary for the image data to be recorded once again or continuously. If the image recording device is an x-ray device, an applied x-ray dose can be drastically reduced. However the image data for improving the quality and accuracy of the overlay image can be re-recorded and updated at predetermined time intervals.

The image recording device can be a tomography device, especially an x-ray computer tomography device, x-ray C-arm device, magnetic resonance tomography device, ultrasound tomography device, Positron Emission Tomography (PET) device or a Single-Photon Emission Computer Tomography (SPECT) device.

The sensor of the magnetic field detection unit can be any sensor for detecting the strength of a magnetic alternating field. Preferably the sensor includes a coil, a Hall sensor, a magneto-restrictive sensor or a Forster sensor or a saturation core magnetometer. It is also possible for the sensor to include a number of for example crossed, coils, Hall sensors and/or suchlike. This enables the strengths of the alternating field to be detected for different spatial directions. The sensor can also a be a sensor chip with a number of integrated Hall elements for detecting a strength of the magnetic field in three spatial directions. The sensor or the magnetic field detection unit can include an electronic circuit for conversion of sensor signals into digital signals able to be processed electronically. A computer can be provided for operation of the sensors, especially for editing and processing the signals.

In a further embodiment of the position fixing system there is provision for the correlation device to include at least one, preferably three, markings, which defines or define (a) predetermined reference point(s) in the first coordinate system. The reference point can be described in the first coordinate system through predetermined reference coordinates. A correlation of the first and second coordinate system can be undertaken especially simply by means of the marking: The magnet can be arranged at the marking, i.e. at the reference coordinates. From the second coordinates of the magnet positioned in this way and the reference coordinates a coordinate transformation rule defining the correlation can be determined in a simple manner between the first and second coordinate system. Preferably the marking is arranged so that this is visible in the first image. The reference coordinates can be determined manually or automatically from the first image or from the image data. This enables an especially precise definition of the reference coordinates and of the correlation of the image data with first coordinates. For an x-ray computer tomography device the markings can be special markings which cause no image artifacts or only minimal image artifacts in the x-ray image.

According to an embodiment of the position fixing system there is further provision for a computer or a control unit to be provided for recording the image data, for control of the image recording device, for control of the rotation of the magnet, for determination of the position of the magnet, for correlation of the first and second coordinate system, for creation of the first and/or second image, for operation of the sensor and/or for positioning of the sensor. Naturally it is also possible to execute further tasks required for operation of the position fixing system by means of the computer, such as storage of the first coordinates, storage and retrieval of the image data and operations of that kind for example.

In accordance with a further measure of the invention an imaging position fixing method for determination of the position of the inventive instrument introduced into a body is provided, with the following steps:
a) Recording image data for creating a first image of a section of the inside of a body by means of an image recording device, with the image data being correlated with first coordinates of a first coordinate system defined by the image recording device,
b) Rotation of the magnet,
c) Detection of a magnetic alternating field created outside a body as a result of the rotation by means a magnetic field determination device featuring at least one sensor,
d) Determining a position of the magnet on the basis of the strength of the recorded alternating field by means of a position determination device, with the position of the magnet being correlated with second coordinates of a second coordinate system defined by the magnetic field detection device,
e) Correlation of the first and second coordinate system by means of a correlation device, and
f) Creation of the first image reproducing the section of the body and of a second image overlaid onto it reproducing the position of the magnet.

The inventive position fixing method allows an especially simple and exact determination of position of the magnet in the body. With the position of the magnet the position of the end section of the instrument can especially be determined. When the instrument is moved in the body, repeated determination of the position of the magnet enables a trajectory of the magnet or of the end section in the body to be determined. The individual positions of the magnet can be reproduced separately from each other or as trajectory in the second image. In a presentation of the trajectory an arrangement of the instrument in the body can be shown in an overlay image reproducing the first image and the second image. An individual position of the magnet can be represented by points, crosses, arrows and other objects of this type. The trajectory can be shown in the form of a highlighted line from the first image, with an instantaneous position of the magnet able to be represented by a particular highlight.

To present the position of the magnet within the body it is not necessary for new image data to be recorded for each change of position of the magnet. For an investigation of the inside of the body by means of a catheter it is sufficient for example to record image data at the beginning of the investigation. In the first image created from the first image data the progress of the position of the magnet or of the end section of the catheter during the investigation can be shown. Use of a first image is in principle possible for as long as the alignment of the body relative to the first coordinate system does not change significantly. The inventive position fixing method in any event means that significantly fewer images need to be recorded. In the case of a recording apparatus operating in accordance with the x-ray method this advantageously results in the applied x-ray dose being able to be drastically reduced.

As regards the advantageous embodiments and advantages of the position fixing method the reader is referred to advantageous embodiments and advantages of the position fixing system which apply analogously to the position fixing method.

### BRIEF DESCRIPTION OF THE DRAWINGS

Exemplary embodiments of the invention are explained below in greater detail with reference to the drawings. The figures show:

FIG. 1 a schematic, enlarged diagram of a first embodiment of the inventive instrument,

FIG. 2 a schematic, enlarged diagram of a second embodiment of the inventive instrument,

FIG. 3 a schematic, enlarged diagram of a third embodiment of the inventive instrument and

FIG. 4 a schematic diagram of the inventive imaging position fixing system.

In the figures the same elements or those with the same function have been labeled with the same reference symbols where it makes sense to do so.

### DETAILED DESCRIPTION OF THE INVENTION

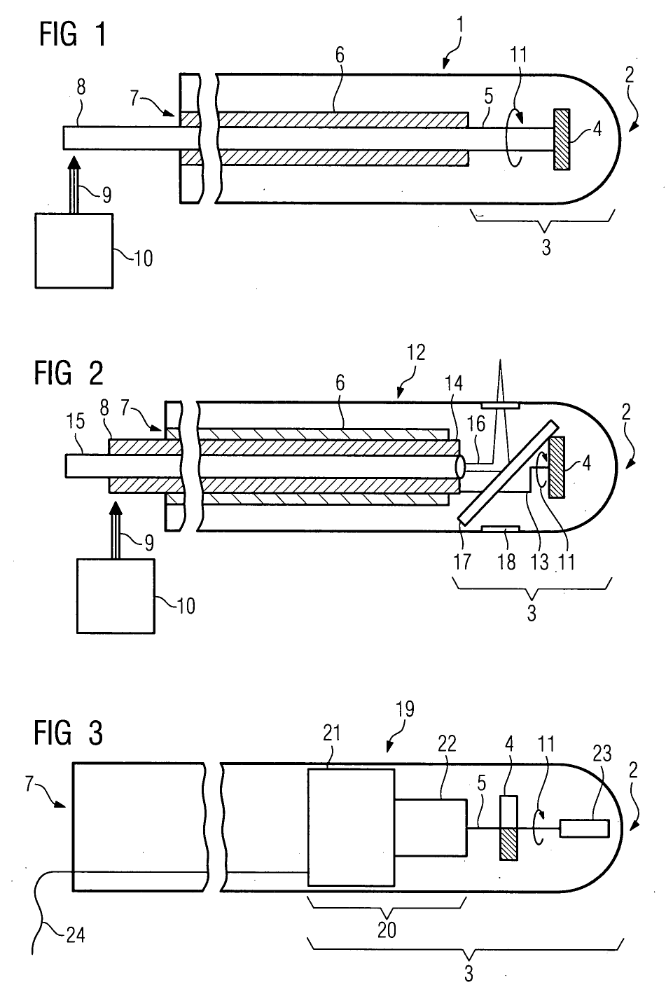

FIG. 1 shows a schematic, enlarged diagram of a first embodiment of the inventive instrument. The instrument concerned is for example a catheter 1 which can be introduced into the body of a living being, especially of a human being, with a free first end 2 for introduction into the body. There is provision for a magnet 4 in the end section 3 containing the free end 2. The magnet 4 is connected to a shaft 5 provided to transmit a rotational movement to the magnet 4 in such a way that a rotation of the magnet 4 creates an alternating magnetic field suitable for locating the magnet's position. Starting from the end section 3, the shaft 5 which can be rotated in a tube 6, is routed through the catheter 1 to a second end 7. A shaft end 8 is brought out at a second end 7 of the catheter 1. The shaft end 8 can be coupled to a drive unit 10 by means of a schematically depicted clutch 9. A direction of rotation of the magnet 4 is indicated by the reference symbol 11.

The function of the catheter 1 is as follows:

The rotational movement of the magnet 4 creates a magnetic alternating field. The strength of the alternating field can be detected by means of sensors. As a result of the reduction of the strength of the alternating field as the distance from the magnet 4 increases, the strength of the alternating field contains information about the position of the magnet 4 relative to the sensor. This information can be used to determine the position of the magnet.

The strength of the alternating field able to be created at a predetermined distance from the magnet 4 increases as the rotation frequency increases. To determine the position of the magnet 4 in the body it is necessary for the alternating field to also be able to be detected with a suitable strength outside the body. The alternating field must be at least strong enough, despite absorption losses in the body, for a threshold value for the sensitivity of the sensor to still be exceeded outside the body. This can be achieved with the catheter 1 in a simple manner by the drive unit 10 being used with a sufficiently high speed of rotation. It can thus be insured that the position of the magnet 4, and thereby of the end section 3 of the catheter 1, is able to be determined with especially high accuracy.

The drive unit 10 can for example be an electric motor with a regulatable speed. To obtain a suitable speed, the drive unit 10 can also include a step-up or a reduction gear.

To transmit the rotational movement from the motor or gear to the shaft 5, the latter can be connected directly to a motor or gear shaft. It is also possible for a floating clutch, claw clutch, leaf clutch, disk clutch, magnetic clutch or suchlike to be used. The clutch can also include runner wheels or rollers which roll on a circumference of the shaft 5 and thus transmit the rotational movement to the shaft 5. The latter allow an especially simple connection of the shaft 5 to the motor or gear, especially for different types and diameters of shaft.

The turning movement is translated by means of the shaft 5 into the rotational movement 11. To avoid variations in the rotational movement 11, which influence the strength of the alternating field and are detrimental to the accuracy of the determination of the position of the magnet 4, the shaft 5 is guided in the tube 6. The inner surface of the tube 6 and the outer surface of the shaft are preferably embodied such that sliding friction between shaft 5 and tube 6 is especially small.

For a rigid catheter 1 the shaft 5 and the tube 6 can be embodied as rigid or flexible devices. For a flexible catheter the shaft 5 and the tube 6 are likewise flexible and are made of a sufficiently fracture-resistant, torsionally stiff, and kink-proof material. The shaft 5 and/or the tube 6 can for example be made of a plastic material.

FIG. 2 shows a schematic, enlarged diagram of a second embodiment of the inventive instrument. The second embodiment involves an OCT catheter 12 for carrying out investigations by means of Optical Coherence Tomography (OCT). In the OCT catheter 12 the magnet 4 is connected to a hollow shaft 14 by means of an attachment element or an attachment arm 13. Starting from the end section 3, the hollow shaft 14, which can be rotated in a tube 6, is routed through the OCT catheter 12 to a second end 7. To transmit the turning movement 11 to the magnet 4, the shaft end 8 can be connected by means of the clutch 9 to the drive unit 10. An optical fiber 15 is routed through the hollow shaft 14. To carry out OCT investigations light 16 can be coupled into the OCT catheter 12 via the optical fiber 15. The coupled-in light 16 can be deflected by means of a mirror 17 arranged on the attachment arm 13 in the end section 3 onto an entry or exit window 18 arranged in the wall of the OCT catheter 12. The light 16 can escape from the OCT catheter 12 via the exit window 18.

The function of the OCT catheter 12 is as follows:

The alternating field is created in a similar way to field creation for the catheter 1 of FIG. 1. The above embodiments are similarly applicable.

Unlike the catheter 1 of FIG. 1 the magnet 4 in OCT catheter 12 is attached to attachment arm 13. The attachment arm 13 is its turn is attached to the hollow shaft 14. This type of attachment allows a rotation of the magnet 4 in the same way as with catheter 1 for creating a suitable magnetic alternating field for determining the position of the magnet 4.

Apart from this investigations by means of Optical Coherence Tomography (OCT) can be carried out using OCT catheter 12. To this end OCT catheter 12 can be introduced into a blood vessel for example. Subsequently light 16 is coupled in via the optical fiber 15. The light 16 exits in the end section 3 from the optical fiber 15 and hits the mirror 17 arranged on the attachment arm 13. The light 16 is deflected by the mirror 17 and exits through the exit window 18 from the catheter and hits the wall of the vessel. A reflection light reflected form the wall of the vessel in the direction of the mirror 17 can be directed via the mirror 17 and the optical fiber 15 to an OCT device not shown in the diagram for detection and processing of the reflection light. The fact that the mirror 17 is arranged on the attachment arm 13 means that the mirror 17 makes the same rotational movement 11 as the magnet 4. As a result of the rotational movement 1 the inside of the vessel wall can be scanned using the light 16. Based on the reflection light an OCT image of the vessel wall can be created which can be used for diagnostic purposes.

The advantage of the inventive OCT catheter 12, as well as giving the option of an especially precise location of the magnet 4, is that only one drive unit 10 is necessary for the magnet 4 and the mirror 17.

FIG. 3 shows a schematic, enlarged diagram of a third embodiment of the inventive instrument. The third embodiment involves an IVUS catheter 19 for carrying out intravascular ultrasound investigations (IVUS investigations). In the IVUS catheter 19 a drive unit 20 is provided in the end section 3. The drive unit 20 includes a motor 21 and gear 22 connected downstream from the motor 21. Attached one after the other to the output shaft 5 of gear 22 are the magnet 4 and an ultrasound converter 23 for carrying out intravascular ultrasound investigations. To supply the motor 21 and ultrasound converter 23 with energy and/or for transmission of signals, at least one first line 24 routed through the IVUS catheter 19 and brought out at the second end 7 is provided.

The function of the IVUS catheter 19 is as follows:

The inventive IVUS catheter 19 differs from the catheter 1 of FIG. 1 and from the OCT catheter 12 in that the motor 21 and the gear 22 are not arranged outside but within the end section 2. Apart from this the alternating field is created in a similar manner to catheter 1 and OCT catheter 12 and the same advantages can be obtained as regards determining the position of the magnet 4.

It is of course also possible to provide a drive unit in the end section with catheter 1 and OCT catheter 12.

With IVUS catheter 19 the transmission of the turning movement via the shaft 5 routed to the second end 7 in the tube 6 is omitted. This makes it possible to avoid the accuracy of the rotation frequency of the magnet 4 being adversely affected by friction resistance of the shaft 5 in the tube 6 and a lengthwise kinking of the IVUS catheter 19. As a result the position of the magnet 4 can be determined precisely.

The ultrasound converter 23 for carrying out intravascular ultrasound investigations is rotated together with the magnet 4.

The first line 24 is provided to supply the drive unit and/or of the ultrasound converter 23 with energy. The first line 24 can also be used for transmission of ultrasound signals from or to the ultrasound converter 23. Signals of the ultrasound converter can also be transmitted via a wireless connection, e.g. a radio connection. For energy supply a source of energy, such as an accumulator, can also be provided in the end section 3.

The motor can for example be an electric motor, stepping motor or a piezoelectric motor etc. The motor 21 can be selected in accordance with the torque for the magnet 4 and ultrasound converter 23 and the dimensions in the end section 3.

The inventive IVUS catheter 19 does not require any connections to external drive elements. As a result the handling of the IVUS catheter 19 can be greatly simplified. Like catheter 1 and OCT catheter 12, IVUS catheter 19 allows an especially simple and exact determination of the position of the magnet 4.

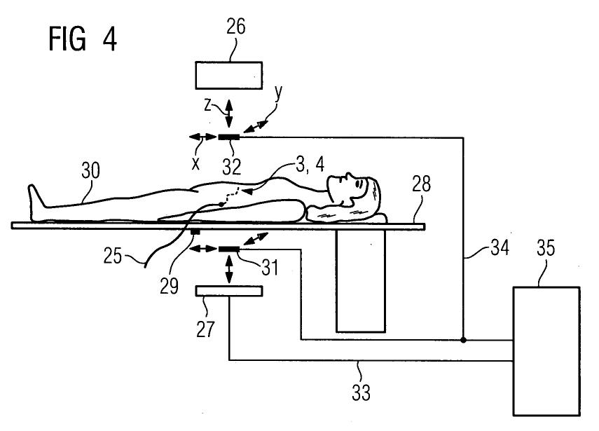

FIG. 4 shows a schematic diagram of the inventive imaging position fixing system. The position fixing system includes an inventive medical instrument 25. The medical instrument 25 involved can for example be a catheter 1 as shown in FIG. 1, an OCT catheter 12 as shown in FIG. 2 or an IVUS catheter 19 as shown in FIG. 3. The position fixing system further features an x-ray device with a x-ray source 26 and a x-ray detector 27 arranged opposite the source. The x-ray source 26 and x-ray detector 27 are arranged on opposite sides of a patient bed 28. Makings 29 are fixed to the patient bed 28 in the recording field of the x-ray device. The markings 29 concerned are specific x-ray markers which can be recorded by the x-ray device and cause no image artifacts or only minimal image artifacts in x-ray images. A patient is accommodated on the patient bed 28, into whose body 30 the instrument 25 is introduced. To detect a magnetic alternating field able to be created by means of a magnet 4 accommodated in the end section 3, a first sensor 31 and a second sensor 32 located opposite the first sensor are provided. To increase the accuracy of determining the position of the magnet, further sensors not shown in the diagram can be provided, the number of which can be 1 to 3, 3 to 10, 10 to 35, 35 to 70, 70 to 100 or greater. The reference symbols x, y, z, designate three spatial directions. The x-ray source 26 and the x-ray detector 27 as well as the first sensor 31 and the second sensor 32 are connected via second 33 or third lines 34 to a computer 35.

The function of the imaging position fixing system is as follows:

First of all image data is recorded by the x-ray device, which includes the x-ray source 26 and the detector 27, to create a first image of a section of the inside of the body 30. The image data can for example involve a series of 2D images. Such 2D image datasets allow a reconstruction of a 3D image of the recorded section. The image data detected by the detector 27 is transmitted via the second line 33 to an image creation device included in the computer 35. From the image data the image creation device can create a first image of the section, e.g. a 2D sectional image or 3D image. The image data recorded for creating each pixel of the first image is correlated with a first coordinate system which is defined by the image recording device. The image data also contains information about the markings 29. The position of the markings 29 can be identified and described with marking coordinates in the first coordinate system. By means of the image recording device the first image with the position of the markings 29 indicated within it can be displayed on a screen not shown in the diagram.

The first image can be used to create an overlay image, which reproduces the section of the inside of the body 30 and within this an exact position of the magnet 4.

To determine the position of the magnet 4, and thereby of the section 3, a magnetic alternating field is created by a rotation of the magnet 4. The magnet 4 can be rotated in a manner similar to the way in which it is rotated in the catheter 1, OCT catheter 12 and IVUS catheter 19 of FIG. 1 to 3. The strength of the alternating field created by the rotation is detected by means of the first 31 and second sensor 32 and if necessary with additional sensors not shown in the figure which are components of a magnetic field detection device. The signals created by the first 31 and second sensor 32 and the further sensors are transmitted via the third line 34 to a position determination device which is a component of a computer 35. On the basis of the signals the position determination device determines the position of the magnet 4 in a conventional manner. In this case the position of the magnet 4 is correlated with a coordinate system defined by the first sensor 31 and second sensor 32.

To create the overlay image it is necessary for the first and second coordinate system to be correlated. For correlation coordinates can additionally be determined in the second coordinate system for the magnet 4 positioned at the marking 29. The marking coordinates and the correlation coordinates describe the same point in space in the first or second coordinate system. Starting from this point a coordinate transformation rule between the first and second coordinate system can be determined, which represents a correlation of the first coordinate system and second coordinate system.

After correlation has been undertaken, the medical instrument, preferably a catheter, a needle and such like, is introduced into the section of the body 30. The position of the magnet 4 can be continuously determined by means of the magnetic field detection device and the position determination device. Provided the orientation of the section relative to the markings 29 does not change significantly, the same first image can be used for the overlay image and within this the instantaneous position of the magnet 4 can be shown. No continuous recording of image data is necessary, so that the applied x-ray dose can be drastically reduced.

An ongoing determination of the position of the magnet 4 can be undertaken as follows:

To determine the position of the magnet 4 location and time-dependent gradients of the alternating field can be dimensioned by means of the first 31 and second sensor 32 and by the further sensors not shown. The position of the magnet 4 can be computed on the basis of the sensor signals. To this end predetermined algorithms can be used, with which for example faults caused by electrical conductors, ferromagnetic objects and such like can also be taken into account.

The overall image can specify the position of the magnet 4 in the form of a simple symbol, e.g. a cross, an arrow and such like. It is also possible for the overlay image to contain a representation of the progress of the medical instrument in the body 30, with end section reproducing the position of the magnet 4. The presentation can for example involve a line which is clearly distinguishable from the first image in color tone.

The computer 35 can be used in the position fixing system for any given control, computation processes and such like. For example the computer 35 can control the actuators, the image recording device and such like and store, edit and process data determined by the first 31 and second sensor 32 and further sensors not shown, as well as the image data.

The x-ray device concerned can be an x-ray computer tomography device or x-ray C-arm device. A magnetic resonance tomography device, ultrasound tomography device, Positron Emission Tomography device, Single-Photon Emission Computer Tomography device or any other imaging device with which imaging data can be recorded for three-dimensional reconstruction of a section of the inside of the body 30 can be used to record image data.

The medical instrument 25 involved can be a catheter, especially an IVUS or OCT catheter, a needle, especially a puncturing needle or biopsy needle, a probe, especially a stomach or bowel probe.

The exemplary embodiments concerned are typical embodiments of the invention. Naturally alternate, similar embodiments are also conceivable within the framework of the invention. For example components of the embodiment can be replaced by alternate components which operate in the same way.

The inventive instrument, position fixing system and position fixing method make it possible to precisely fix the position of a magnet 4 provided in an end section 3. Furthermore the inventive magnet 4 attached to a rotation drive can be integrated in a simple manner into known, especially medical, devices. This applies especially to catheters with a shaft 5 which is present in any event. This allows an especially simple option for fixing the position of the magnet 4 or end section 3 to be provided. Apart from this it is possible, with recording systems in which the body is subjected to damaging radiation when the image data is recorded, to greatly reduce the radiation load.

1.-23. (canceled)

24. An instrument used in a medical examination, comprising:
an end section comprising a free end that inserts into a body under the medical examination;
a magnet that is arranged in the end section and determines a position of the instrument within the body; and
a rotation drive that is connected to the magnet and rotates the magnet.

25. The instrument as claimed in claim 24, wherein the magnet is a permanent magnet or an electromagnet.

26. The instrument as claimed in claim 24,
wherein the rotation drive comprises a shaft and a drive unit,
wherein the magnet is directly attached to the shaft or arranged on an attachment arm fixed to the shaft, and
wherein the shaft transmits a rotation movement from the drive unit to the magnet.

27. The instrument as claimed in claim 26, wherein the shaft is a flexible shaft and comprises a plastic material.

28. The instrument as claimed in claim 26,
wherein the shaft is routed in a tube,
wherein the tube is a flexible tube, and
wherein the flexible tube comprises a plastic material.

29. The instrument as claimed in claim 26, wherein the drive unit is arranged outside the instrument and connected to the shaft by a clutch.

30. The instrument as claimed in claim 26, wherein the drive unit is arranged in the end section of the instrument.

31. The instrument as claimed in claim 26, wherein the drive unit comprises a motor selected from the group consisting of: an electric motor, a stepping motor, a piezoelectric motor, a turbine motor, a hydraulic motor, and a pneumatic motor.

32. The instrument as claimed in claim 24, wherein the rotation drive comprises a gear.

33. The instrument as claimed in claim 24, wherein the rotation drive rotates:
an ultrasound converter for performing an intravascular ultrasound examination in an area of the end section, or
a mirror for performing an optical coherence tomography examination in an area of the end section.

34. The instrument as claimed in claim 24,
wherein the instrument is selected form the group consisting of: a catheter, a needle, and a probe, and
wherein the catheter is a IVUS or OCT catheter, the needle is a puncturing or a biopsy needle, and the probe is a stomach or a bowel probe.

35. An imaging positioning system for determining a position of an instrument introduced into a body under examination, comprising:
an image recording device that records image data of a section of an inside of the body, wherein the image data being correlated with a first coordinate system of the image recording device;
a magnet that is arranged in an end section of the instrument introduced into the body and rotates in the body by a rotation drive connected to the magnet;
a magnetic field detection device that is arranged outside the body for detecting a strength of a magnetic alternating field created by the rotation of the magnet in the body; and
a computer that:
determines a position of the magnet based on the strength of the detected magnetic alternating field, wherein the position of the magnet being correlated with a second coordinate system of the magnetic field detection device,
correlates the first coordinate system with the second coordinate system,
creates a first image from the recorded image data of the section, and
creates a second image by overlaying the position of the magnet on the first image.

36. The imaging positioning system as claimed in claim 35, wherein the image recording device is selected from the group consisting of: an x-ray computer tomography device, an x-ray C-arm device, a magnetic resonance tomography device, an ultrasound tomography device, a Positron Emission Tomography device, and a Single-Photon Emission Computer Tomography device.

37. The imaging positioning system as claimed in claim 35, wherein the magnetic field detection device comprises a sensor selected from the group consisting of: a coil, a Hall sensor, a magneto-restrictive sensor, a Forster sensor, and a saturation core magnetometer.

38. The imaging positioning system as claimed in claim 35, wherein the computer further controls the image recording device, the rotation drive, and the magnetic field detection device.

39. The imaging positioning system as claimed in claim 35, further comprising a marker that define a predetermined reference point in the first coordinate system for the correlation.

40. A method for determining a position of an instrument introduced into a body of a patient, comprising:
recording an image data of the body by an image recording device, wherein the image data is correlated with a first coordinate system of the image recording device;
arranging a magnet in an end section of the instrument introduced into the body;
rotating the magnet;
detecting a strength of a magnetic alternating field created by the rotation of the magnet;
determining a position of the magnet by a magnetic field detection device based on the strength of the detected magnetic alternating field, wherein the position of the magnet is correlated with a second coordinate system of the magnetic field detection device;
correlating the first coordinate system with the second coordinate system;
creating a first image from the recorded image data of the body; and
creating a second image by overlaying the position of the magnet on the first image for a medical examination of the patient.

41. The method as claimed in claim 40, wherein the strength of the magnetic alternating field is detected by a sensor selected from the group consisting of: a coil, a Hall sensor, a magneto-restrictive sensor, a Förster sensor, and a saturation core magnetometer.

42. The method as claimed in claim 40, wherein the first coordinate system is correlated with the second coordinate system based on a predetermined pixel in the first or the second coordinate system.

43. The method as claimed in claim 40, wherein the steps of recording, rotating, detecting, determining, correlating, and creating the first and the second images are controlled by a computer.
